(19) United States
(12) Patent Application Publication
(10) Pub. No.: US 2025/0050069 A1
(43) Pub. Date: Feb. 13, 2025

(54) Catheter Stabilization Device

(71) Applicant: Solventum Intellectual Properties Company, Maplewood, MN (US)

(72) Inventor: Alan R. Dombrowski, Woodbury, MN (US)

(21) Appl. No.: 18/719,507

(22) PCT Filed: Dec. 1, 2022
(86) PCT No.: PCT/IB2022/061656

§ 371 Date: Jun. 13, 2024

Related U.S. Application Data
(60) Provisional application No. 63/290,927, filed on Dec. 17, 2021.

(51) Int. Cl. A61M 25/02

(52) U.S. Cl. CPC A61M 25/02; A61M 2025/0253

(57) ABSTRACT

A catheter attachment system includes an adhesive attachment layer and a polymeric hardgood article 100 for holding the catheter. The polymeric hardgood is a hinged article that contains two interlocking members. The first member is a holding member 120 with a securement surface designed to hold the hub of a catheter. The securement surface is a conformable layer that has a high coefficient of friction with a value of 0.5 or higher. The second member 130 is a cover member hingably linked to the holding member, where the cover member is designed to contact at least a portion of the securement surface of the holding member when closed, to hold the hub of a catheter.

### SUMMARY

Disclosed herein are attachment systems for attaching a catheter to a patient, and medical constructions that include the attachment system.

In some embodiments, the attachment system comprises an adhesive attachment layer with a first major surface and a second major surface, the first major surface comprising an adhesive surface suitable for attachment to mammalian skin, and a polymeric hardgood article for holding a catheter. The polymeric hardgood comprises a base layer with a first major surface and a second major surface, where the first major surface is in contact with the second major surface of the adhesive attachment layer and a hinged article that contains at least two interlocking members. The hinged article comprises a first member that is a holding member comprising two major surfaces wherein the first major surface of the holding member is in contact with the second major surface of the base layer, or wherein the first major surface of the holding member is the base layer, and the second major surface of the holding member comprises a securement surface designed to hold a hub of a catheter. The second major surface of the holding member comprises at least a conformable layer that has a high coefficient of friction with a value of 0.5 or higher. The second member is a cover member hingably linked to the holding member, where the cover member comprises two major surfaces wherein the first major surface of the cover member is designed to contact at least a portion of the second major surface of the holding member when closed, such that when closed the hinged article holds the hub of a catheter. The first major surface of the cover member comprises a conformable layer.

Also disclosed herein are medical constructions comprising a surface comprising the mammalian skin of a patient, a catheter attached to the patient, where the catheter comprises a hub, and an attachment system attached to the mammalian skin of the patent and holding the catheter hub. The attachment system is described above.

### BRIEF DESCRIPTION OF THE DRAWINGS

The present application may be more completely understood in consideration of the following detailed description of various embodiments of the disclosure in connection with the accompanying drawings.

FIGS. 1A and 1B re perspective views of a hardgood article of this disclosure.

FIG. 2 is a perspective view of another hardgood article of this disclosure.

FIGS. 3A-3C are perspective views of a catheter stabilization device of this disclosure in use.

FIGS. 4A and 4B are perspective views of another catheter stabilization device of this disclosure in use.

In the following description of the illustrated embodiments, reference is made to the accompanying drawings, in which is shown by way of illustration, various embodiments in which the disclosure may be practiced. It is to be understood that the embodiments may be utilized and structural changes may be made without departing from the scope of the present disclosure. The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

Various medical devices are attached to a patient. For example, tubing, monitors, sensors, or catheters are secured to skin. To limit irritation, dislodgement, and potential exposure to infection, the medical devices should be securely attached to the patient. Adhesives and adhesive tapes are commonly used to secure devices to skin. Very strong adhesives can cause trauma to skin upon removal. A very gentle adhesive will remove from the skin easily but might not have sufficient strength to secure the medical device. Therefore, there is a need for medical adhesive articles that adhere strongly and yet are removable without causing skin damage or pain.

Among the recent developments are the use of stretch removability to permit removal of the adhesive article from the skin. In these articles, a film backing is used that has an extensible substrate with a securing adhesive that can secure to a surface and a tab to stretch the extensible substrate to remove the adhesive from the underlying surface. Stretching the extensible substrate will release the securing adhesive from the underlying surface easily and without trauma. Therefore, a strong adhesive can be used.

This mechanism is known as “stretch release”. Stretch release tapes are known for use in securing items to surface, such as walls. For example, 3M COMMAND tape is a stretch release tape with a foam substrate coated on both surfaces with a strong adhesive. In U.S. Patent Ser. No. 62/910,667 filed on Oct. 4, 2019 such a stretch releasable medical tape article is described.

Among the medical articles that are attached to patients are vascular devices. Vascular devices are devices that are inserted into veins for diagnostic or therapeutic reasons, such as blood sampling, central venous pressure readings, administration of medication, fluids, total parenteral nutrition (TPN) and blood transfusions. Among vascular devices are central venous access devices (CVADs) or central venous catheters (CVCs), devices that are inserted into the body through a vein to enable the administration of fluids, blood products, medication and other therapies to the bloodstream. CVADs can be inserted into the subclavian or jugular vein (implanted ports, tunneled catheters), or can be inserted into one of the peripheral veins of the upper extremities, called peripherally inserted central catheters (PICCs).

Catheters of various types (e.g. venous, arterial) are routinely placed in patients in hospitals and are typically secured using sutures, tape, a dressing or a securement device. Movement of an inserted catheter can lead to irritation of the vein and other complications, requiring premature removal of the catheter and insertion of the catheter in a different vein. This results in a waste of resources in both medical supplies and labor, as well as extended patient care and discomfort. Further, since each patient has a limited number of catheter insertion sites, it is undesirable to repeatedly replace a catheter at a new insertion site on a patient this process expends the available insertion sites.

It is desirable to have a catheter attachment device that is “universal”, meaning that it is able to securely hold a wide range of different catheter types. There are many different manufacturers of catheter hubs and these hub sizes vary, and hub sizes vary even within the same manufacturer, therefore a securement device that can accommodate a wide variety of catheters are preferred by clinicians. Typically, these products often utilize aggressive adhesives that must be removed using an adhesive-remover or alcohol. Therefore, the need remains for an attachment system for a wide range of vascular devices that is easy to use, provides stable securement, and is gentle to remove without adverse reactions to skin.

Disclosed herein is a system including a hardgood design that allows for adequate securement of many different catheters and a stretch-removable film layer that enables skin-friendly removal of the system from the patient. The attachment system comprises an adhesive attachment layer and a polymeric hardgood article for holding a catheter.

The term “adhesive” as used herein refers to polymeric compositions useful to adhere together two adherends. Examples of adhesives are pressure sensitive adhesives and gel adhesives.

Pressure sensitive adhesive compositions are well known to those of ordinary skill in the art to possess properties including the following: (1) aggressive and permanent tack, (2) adherence with no more than finger pressure, (3) sufficient ability to hold onto an adherend, and (4) sufficient cohesive strength to be cleanly removable from the adherend. Materials that have been found to function well as pressure sensitive adhesives are polymers designed and formulated to exhibit the requisite viscoelastic properties resulting in a desired balance of tack, peel adhesion, and shear holding power. Obtaining the proper balance of properties is not a simple process.

As used herein, the term “gel adhesive” refers to a tacky semi-solid crosslinked matrix containing a liquid or a fluid that is capable of adhering to one or more substrates. The gel adhesives may have some properties in common with pressure sensitive adhesives, but they are not pressure sensitive adhesives. “Hydrogel adhesives” are gel adhesives that have water as the fluid contained within the crosslinked matrix.

The term “(meth)acrylate” refers to monomeric acrylic or methacrylic esters of alcohols. Acrylate and methacrylate monomers or oligomers are referred to collectively herein as “(meth)acrylates”. Materials referred to as “(meth)acrylate functional” are materials that contain one or more (meth)acrylate groups.

The terms “siloxane-based” as used herein refer to polymers or units of polymers that contain siloxane units. The terms silicone or siloxane are used interchangeably and refer to units with dialkyl or diaryl siloxane (—SiR₂O—) repeating units.

The terms “room temperature” and “ambient temperature” are used interchangeably to mean temperatures in the range of 20° C. to 25° C.

The term “adjacent” as used herein when referring to two layers means that the two layers are in proximity with one another with no intervening open space between them. They may be in direct contact with one another (e.g. laminated together) or there may be intervening layers.

The terms “polymer” and “macromolecule” are used herein consistent with their common usage in chemistry. Polymers and macromolecules are composed of many repeated subunits. As used herein, the term “macromolecule” is used to describe a group attached to a monomer that has multiple repeating units. The term “polymer” is used to describe the resultant material formed from a polymerization reaction.

As used herein, the terms “microstructured configuration” refers to a surface that contains microstructures and the term “microstructuring” refers to imparting to a surface a microstructured configuration.

As used herein, the term “microstructure” means the configuration of features wherein at least 2 dimensions of the features are microscopic. The topical and/or cross sectional view of the features must be microscopic. Microstructures are features that are deliberately imparted to the surface and are different from the natural surface roughness of the surface.

As used herein, the term “microscopic” refers to features of small enough dimension so as to require an optic aid to the naked eye when viewed from any plane of view to determine its shape. One criterion is found in Modern Optic Engineering by W. J. Smith, McGraw-Hill, 1966, pages 104-105 whereby visual acuity, “ . . . is defined and measured in terms of the angular size of the smallest character that can be recognized.” Normal visual acuity is considered to be when the smallest recognizable letter subtends an angular height of 5 minutes of arc on the retina. At a typical working distance of 250 mm (10 inches), this yields a lateral dimension of 0.36 mm (0.0145 inch) for this object.

Disclosed herein are attachment systems for attaching a catheter to a patient. The attachment system comprises an adhesive attachment layer and a polymeric hardgood article for holding a catheter. The adhesive attachment layer has a first major surface and a second major surface, the first major surface comprises an adhesive surface suitable for attachment to mammalian skin and the second major surface comprises an adhesive surface that is contact with and adhered to the polymeric hardgood article. The polymeric hardgood comprises a base layer and a hinged article. The base layer has a first major surface and a second major surface, where the first major surface is in contact with the second major surface of the adhesive attachment layer. The hinged article may include the base layer or it may be detachable from the base layer. The hinged article contains at least two interlocking members, a first member that is a holding member and a second member that is a cover member hingably linked to the holding member. The holding member comprises two major surfaces wherein the first major surface of the holding member is in contact with the second major surface of the base layer, the first major surface of the holding member is the base layer. The second major surface of the holding member comprises a securement surface designed to hold a hub of a catheter. The second major surface of the holding member comprises at least a conformable layer that has a high coefficient of friction with a value of 0.5 or higher. The cover member comprises two major surfaces wherein the first major surface of the cover member is designed to contact at least a portion of the second major surface of the holding member when closed, such that when closed the hinged article holds the hub of a catheter. The first major surface of the cover member comprises a conformable layer that has a high coefficient of friction with a value of 0.5 or higher.

The adhesive attachment layer comprises a multi-layer stretch removable article. In some embodiments, the multi-layer stretch removable article comprises a multi-layer extensible backing substrate, and two adhesive layers. In some embodiments, the multi-layer extensible backing substrate comprises at least three layers. These layers comprise a first plastic skin layer with a first major surface and a second major surface, a core elastomeric layer with a first major surface and a second major surface, and a second plastic skin layer with a first major surface and a second major surface.

The core elastomeric layer is in contact with the second major surface of the first plastic skin layer and the first major surface of the second plastic skin layer. Examples of suitable multi-layer extensible backing substrates are described in U.S. Patent Ser. No. 62/910,667. In the multi-layer extensible backing substrate, the core comprises an elastomeric material. Elastomeric means that the material exhibits at least some ability to return at least in part to its original shape or size after forces causing the deformation are removed. Examples of elastomeric material include elastomeric polymer, SEBS, SEPS, SIS, SBS, polyurethane, ethyl vinylacetate (EVA), ethyl methyl acrylate (EMA), ultra low linear density polyethylene (ULLDPE), hydrogenated polypropylene, and combinations or blends thereof.

The skin layers of the multi-layer extensible backing substrate may be the same or different, typically they are the same. In some embodiments, the skin layers comprise a plastically deforming material. Plastic deformation means that the material undergoes a permanent change in shape or size when subjected to a stress exceeding a particular value (the yield value). Examples of plastically deforming materials include polypropylene, polyethylene, high density polyethylene (HDPE), low density polyethylene (LDPE), linear low density polyethylene (LLDPE), polyurethane, ethyl vinylacetate (EVA), ethyl methyl acrylate (EMA), and combinations or blends thereof.

The core layer and skin layers can be bonded to one another using any suitable mechanism including, for example, coextruding the core and the skin layer(s), co-molding, extrusion coating, joining through an adhesive composition, joining under pressure, joining under heat, and combinations thereof.

The extensible substrate has a low Fn Modulus. The Fn Modulus is the force required to elongate the extensible substrate. The Fn Modulus is the force (F) required to elongate the test specimen a specified percent (n). Fn Modulus is usually recorded for elongation of 10%, 50%, and 100% for comparison of films.

Particularly suitable extensible substrates for application on skin has a F(50%) Modulus less than 20 N per 25.4 mm width. In some embodiments, the extensible substrate has a F(50%) Modulus less than 10 N per 25.4 mm width at 50% strain, or even a F(50%) Modulus less than 5 N per 25.4 mm width.

Elongation is the maximum percent of strain reached by a test sample to the point of breakage. The extensible substrate has a low elongation to minimize the distance needed to stretch and pull the extensible substrate. A very high elongation means the extensible substrate will have a high distance it stretches before releasing from the surface. Excessive stretching is an inconvenience in settings where space is constrained. Modulus and elongation typically are measured under ambient conditions (25° C. and 50% relative humidity) by a method based upon PSTC-31, ASTM D882 and D3759 Test Methods. The test can be performed on a constant rate of extension/tensile tester (such as an Instron, Zwick or equivalent). Typically, the extensible substrate 110 has an elongation of less than 600%. In some embodiments, the extensible substrate has an elongation of less than 500%, an elongation less than 300%, or even an elongation of at least 100%.

It is desirable that a majority of the deformation of the extensible substrate is plastic deformation and that its elastic deformation is minimized. Elasticity is the ability of a deformed material body to return to its original shape and size when the forces causing the deformation are removed. In contrast, plasticity is the property of a solid body whereby it undergoes a permanent change in shape or size when subjected to a stress exceeding a particular value (the yield value). A highly elastic film may present a hazard during stretching to remove the film from a surface because as the film stretches, a significant amount of energy is stored in it. If the stretching film is not secured, upon the release of the film from the surface this energy will accelerate the film, and any attached device, toward the hand stretching the film, possibly resulting in injury or a broken device.

Plastic deformation is calculated using the original length of the extensible substrate (Lo), the length the extensible substrate reached under applied stress (Ls) and the length the extensible substrate relaxed to after the stress was removed (Lr).

% Plastic deformation=(Lr−Lo)/(Ls−Lo)×100

For example, a one-inch (Lo) length of film stretched to five inches (Ls), which relaxes to a length of four inches (Lr) when the applied stress is removed, has a plastic deformation of 75%. ((4−1)/5−1)×100).

Plastic deformation of the extensible substrate reduces the energy released upon the removal of stress, since the energy input to extensible substrate is consumed by the re-ordering the film morphology rather than stored as potential energy in elastic structures. Less stored energy improves safety during the removal of devices held in place with an extensible substrate. The combination of low modulus and large degree of plastic deformation makes one-handed stretch and removal of the extensible substrate from the surface possible, without the hazard of the extensible substrate from being snapped painfully into the removing hand.

The extensible substrate has a plastic deformation of at least 50%. In some embodiments, the extensible substrate has a plastic deformation of at least 60%, or even at least 70%.

The multi-layer extensible backing substrate may further comprise a tab at a perimeter portion of the extensible substrate. This tab is non-tacky and is designed to be grasped by a user to affect the stretch removal of the adhesive article. In some embodiments, the tab is a portion of and edge of the film backing that is free of adhesive. In other embodiments, the tab is a cover over the securing adhesive to create a portion of the first major surface that is free of a tacky adhesive. Therefore, a user can easily pull the extensible substrate from the underlying surface to remove the extensible substrate from the surface to which it is adhered.

In some embodiments, the stretch removable article further comprises a second tab on the opposite end of the article from the first tab. The second tab may be the same or different from the first tab.

The adhesive attachment layer also comprises two adhesive layers, disposed on the multi-layer extensible backing substrate. These adhesive layers are described as a first adhesive layer and second adhesive layer. The first adhesive layer is in contact with the first plastic skin layer, and the first adhesive layer is suitable for attachment to mammalian skin. The second adhesive layer is in contact with the third plastic skin layer, and the second adhesive layer attaches the multi-layer stretch removable article to the polymeric hard good article.

The first adhesive layer may be continuous or discontinuous and is suitable for adhering the stretch removable article to human skin. Typically, the securing adhesive is a pressure sensitive adhesive. Among the suitable classes of pressure sensitive adhesives include rubber-based adhesives, siloxane-based adhesives, and (meth) acrylate-based adhesives. In some embodiments, the adhesive can include tackified rubber adhesives, such as natural rubber; olefins; siloxanes, such as silicone polyureas: synthetic rubber adhesives such as polyisoprene, polybutadiene, and styrene-isoprene-styrene, styrene-ethylenebutylene-styrene and styrene-butadiene-styrene block copolymers, and other synthetic elastomers; and tackified or untackified (meth) acrylate adhesives such as copolymers of isooctylacrylate and acrylic acid, which can be polymerized by radiation, solution, suspension, or emulsion techniques: polyurethanes: silicone block copolymers: and combinations of the above. The adhesive can be, for example, any of the adhesives described in PCT Patent Publication Nos. WO 2015/035556, WO 2015/035960, and US 2015/034104. In some embodiments, the adhesive includes additives such as tackifiers. Some exemplary tackifiers include polyterpene, terpene phenol, rosin esters, and/or rosin acids.

In some embodiments, the first adhesive layer may be a gel adhesive. Gel adhesives are tacky semi-solid crosslinked matrix containing a liquid or a fluid that is capable of adhering to one or more substrates. The gel adhesives may have some properties in common with pressure sensitive adhesives, but they are not pressure sensitive adhesives.

The first adhesive layer may comprise a variety of additives as long as the additives do not interfere with the adhesive properties of the adhesive layer. Particularly suitable additives include anti-microbial additives, rendering the first adhesive layer into an anti-microbial adhesive layer.

A wide range of anti-microbials are suitable for use in the first adhesive layer. Suitable anti-microbial additives include benzalkonium chloride, copper, silver, quaternary ammonium compounds, polyhexamethylene biguanide, chlorhexidine gluconate, fatty acid monoesters.

The second adhesive layer may be continuous or discontinuous and attaches the multi-layer stretch removable article to the polymeric hard good article. Typically, this layer is a pressure sensitive adhesive. The second adhesive layer is often different from the first adhesive layer, since the second adhesive layer is not a mammalian skin adhesive. A wide range of pressure sensitive adhesives are suitable. Pressure sensitive adhesives useful for the second adhesive layer include tackified natural rubbers, synthetic rubbers, tackified styrene block copolymers, polyvinyl ethers, acrylics, poly-o-olefins, and silicones.

The first adhesive layer may comprise a variety of additives as long as the additives do not interfere with the adhesive properties of the adhesive layer. Particularly suitable additives include anti-microbial additives, rendering the first adhesive layer into an anti-microbial adhesive layer.

A wide range of anti-microbials are suitable for use in the first adhesive layer. Suitable anti-microbial additives include benzalkonium chloride, copper, silver, quaternary ammonium compounds, polyhexamethylene biguanide, chlorhexidine gluconate, fatty acid monoesters.

The attachment system also comprises a hardgood article. The polymeric hardgood comprises a base layer and a hinged article. In some embodiments, the base layer is part of the hinged article, in other embodiments the base layer is a separate unit and the hinged article is detachable from the base layer. Each of these embodiments is described below.

The base layer has a first major surface and a second major surface, where the first major surface is in contact with the second major surface of the adhesive attachment layer described above. In the embodiments where the base layer is a separate unit, the second major surface of the base layer is a surface to which the hinged article is detachably adhered. In other embodiments, the base layer is part of the hinged article.

In the embodiments where the base layer is a separate unit, the base layer is typically a planar article. In these embodiments, the base layer can remain attached to the patient, while the hinged article can be attached and detached as desired. The base layer includes an detachable attachment system for holding the hinged article to the base layer. Typically, this detachable attachment system is a mechanical system such as snaps, clasps, and the like.

The hinged article, whether it includes the base layer or is separate from the base layer, is similar in design. The hinged article contains at least two interlocking members, a first member that is a holding member and a second member that is a cover member hingably linked to the holding member. The holding member comprises two major surfaces, a first major surface of the holding member is in contact with the second major surface of the base layer, or the first major surface of the holding member is the base layer. The second major surface of the holding member comprises a securement surface designed to hold a hub of a catheter. The second major surface of the holding member comprises at least a conformable layer that has a high coefficient of friction with a value of 0.5 or higher. The cover member comprises two major surfaces, where the first major surface of the cover member is designed to contact at least a portion of the second major surface of the holding member when closed, such that when closed the hinged article holds the hub of a catheter. The first major surface of the cover member comprises a conformable layer that has a high coefficient of friction with a value of 0.5 or higher.

In some embodiments, the holding member securement surface is not a single surface but is subdivided into different subsections. This can be useful to help hold a catheter hub, to provide a nesting space for the hub. In many embodiments, the catheter hub has a thicker central section and two wing sections attached to this thicker central section. To accommodate these sections of the catheter hub, in some embodiments the securement surface comprises at least three subsections. In some embodiments, the three sections comprise a first (edge) section, a second (central) section, and a third (edge) section such that the second section is a depression relative to the first section and the third section. This depression forms the nesting space for the thicker central portion of the hub and the two edge sections form locations to secure the wing sections of the hub. The subsections of the securement surface may be prepared from the same material or materials as described below, or the subsections may be prepared from different materials.

The securement surface of the holding member besides being divided into subsections, may also be a multi-layered surface. Because the holding member is a hinged article that is designed to hold a catheter hub when closed, it is desirable that the surfaces of the holding member be conformable. This permits the surfaces to flex when holding the hub to hold the hub securely. Additionally, to prevent movement of the secured hub it is desirable that the securement surfaces have a high coefficient of friction. In some embodiments, a single material provides these two features, conformability and high coefficient of friction, in other embodiments the securement surface comprises at least two layers where the two layers combined provide these features. In some embodiments, the securement surface comprises two layers where the first layer is disposed on the holding member and comprises a conformable layer, and a second layer disposed on the first layer and comprises a high coefficient of friction surface. In this way the hub contacts a high coefficient of friction surface that is undergirded by a conformable material.

A wide range of materials are suitable for the first conformable layer. Among the suitable materials are foam materials, rubber polymers, and the like. Similarly, a wide range of materials are suitable for the second high coefficient of friction surface. Besides a variety of materials, the high coefficient of friction second layer can have a variety of configurations. In some embodiments, the high coefficient of friction second layer is essentially smooth, in other embodiments, the high coefficient of friction second layer has a microstructured configuration. Microstructuring of the surface that contacts the catheter hub can provide increased securement of the hub. Examples of suitable microstructured features that increase securement of the hub include those described in U.S. Pat. Nos. 7,703,169, 6,610,382, and 6,904,615. Typically, microstructured surfaces are prepared by preparing a microstructured mold and contacting the surface material to the mold at an elevated temperature to impart the microstructuring to the surface. The microstructured surface has a series of protrusions and depressions, where the protrusions generally have an aspect ratio (the ratio of height to width) of at least 1.25.

The first major surface of the cover member that is hingably linked to the holding member can also be prepared from a wide range of materials. Like the securement surface of the holding member, the first major surface of the cover member is conformable and has a high coefficient of friction. In this way the bud of the catheter is securely held because it is contacted by two surfaces that have a high coefficient of friction. The first major surface of the cover member may be comprised of the same material as the securement surface of the holding member, or it may be comprised of a different material. Generally, the first major surface of the cover member is not subdivided into subsections like the securement surface, because it is typically desirable that the first major surface of the cover member be continuous to provide securement of the catheter hub when the hinged article is closed. Like the securement surface, the first major surface of the cover member may be a single material, or it may be a multi-layer surface. In some embodiments, the first major surface of the cover member comprises two layers where the first layer is disposed on the holding member and comprises a conformable layer, and a second layer disposed on the first layer and comprises a high coefficient of friction surface. In this way the hub is held in place by two high coefficient of friction surfaces that are undergirded by a conformable material.

Also disclosed are medical constructions. In some embodiments, the medical construction comprises a surface comprising the mammalian skin of a patient, a catheter attached to the patient, wherein the catheter comprises a hub; and an attachment system attached to the mammalian skin of the patent and holding the catheter hub. The attachment systems are described in detail above. As described above, the attachment system comprises an adhesive attachment layer attached to mammalian skin and a polymeric hardgood article for holding a catheter hub. The polymeric hardgood articles include a base layer and a hinged article.

In some embodiments, the medical construction comprises a multi-layer stretch removable article as described in detail above. As mentioned above, the adhesive attachment layer further comprises one or more tabs at a perimeter portion of the extensible substrate of the adhesive attachment layer. In some embodiments, it is desirable that one of more of these tabs is folded into the polymeric hardgood article. In this way the tabs are out of the way while the device is in use and if additional manipulations are carried out with the catheter.

In some embodiments, the medical construction further comprises a cover film article covering at least a portion of the attachment system and a portion of the mammalian skin. The cover film article can provide supportive attachment and protect the vascular device from exposure to the environment. This can help to keep the site of the catheter attachment clean and free from contaminants.

In some embodiments, the cover film article comprises a cover film with a first major surface and a second major surface, and a third adhesive layer, where the third adhesive layer has a first major surface and a second major surface. The second major surface of the third adhesive layer is in contact with the first major surface of the cover film, and the first major surface of the third adhesive layer is in contact with the second major surface of the polymeric hard good article and the mammalian skin.

Typically, it is desirable for the film of the film article to be optically transparent or even optically clear such that the underlying attachment article can be viewed and monitored. Suitable film materials include standard film-forming materials such as polyester, polyurethane, (meth) acrylate, polyolefin, or a combination thereof. Generally, the film has a thickness of greater than 25 micrometers (1 mil).

The third adhesive layer is typically a pressure sensitive adhesive. In some embodiments, it is desirable that the third adhesive layer be optically transparent or even optically clear such that the underlying attachment article can be viewed and monitored. A wide range of suitable adhesives are suitable. Pressure sensitive adhesives useful for the second adhesive layer include tackified natural rubbers, synthetic rubbers, tackified styrene block copolymers, polyvinyl ethers, acrylics, poly-alpha-olefins, and silicones.

The current disclosure may be more fully understood in light of the figures showing embodiments of the current disclosure.

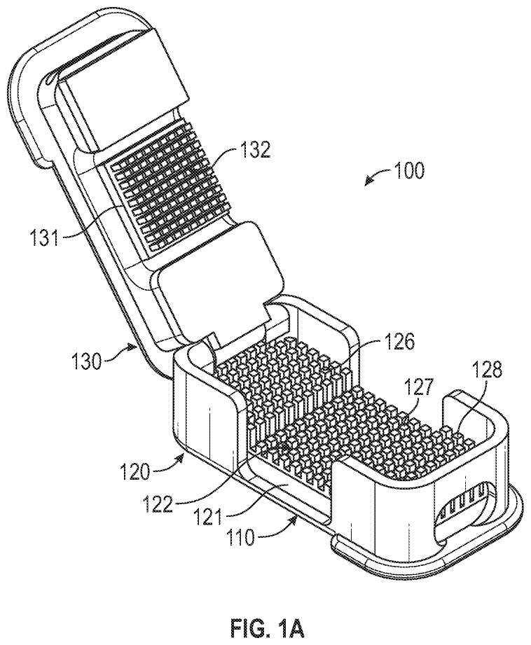

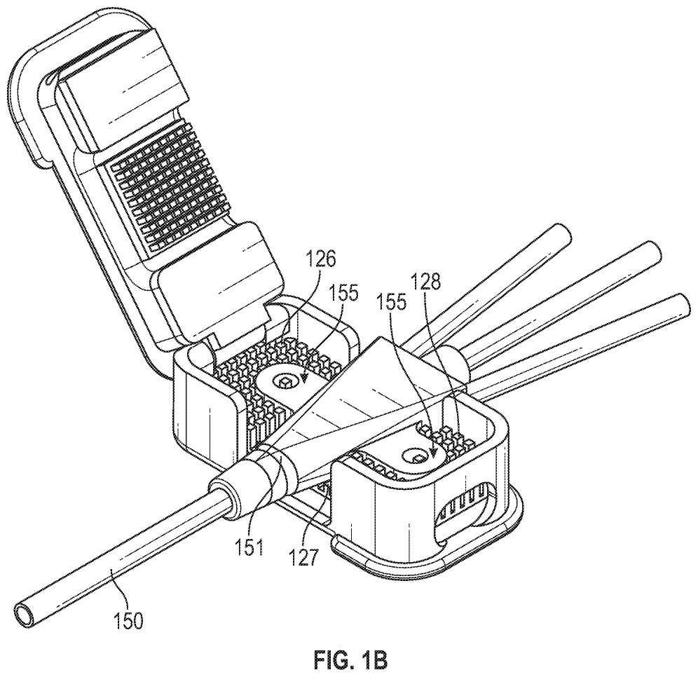

FIG. 1A shows a perspective view of hardgood article 100. Hardgood article 100 has base layer 110, Holding member 120, and cover member 130. Holding member 120 has conformable layer 121 with high coefficient of friction surface 122. Cover member 130 has conformable layer 131 with high coefficient of friction surface 132. The conformable layer 121 of Holding member 120 has segments 126, 127, and 128. Segment 127 is depressed relative to segments 126 and 128.

FIG. 1B show hardgood article 100 with a catheter and hub article disposed within. Catheter 150 is attached to catheter hub 151 with catheter hub wings 155. The hub 151 is disposed in depressed segment 127 and wings 155 are disposed on segments 126 and 128.

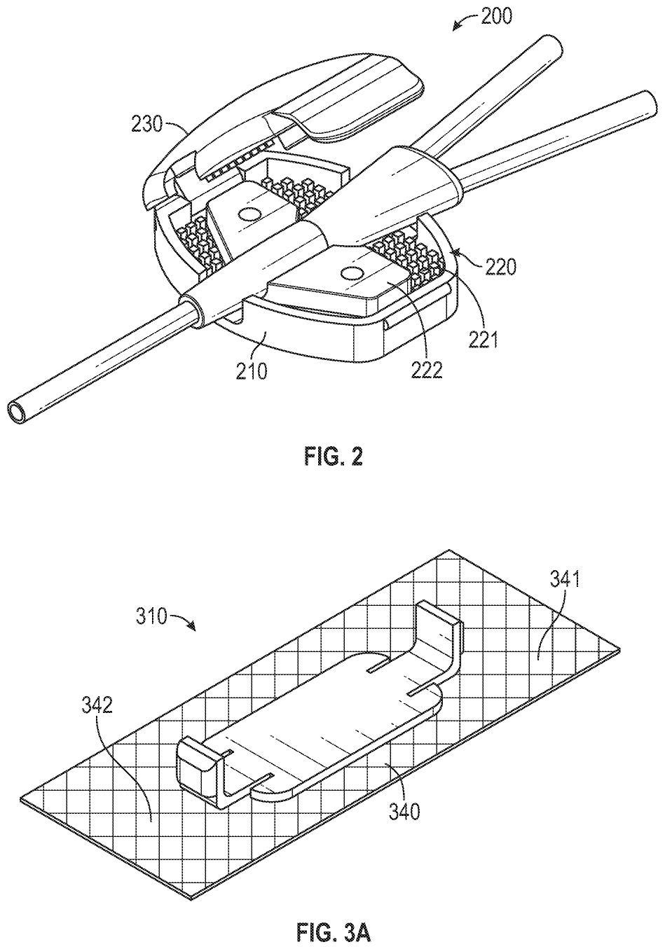

FIG. 2 shows a perspective view of another hardgood article 200. Hardgood article 200 has base layer 210, holding member 220, and cover member 230. Holding member 220 has conformable layer 221 with high coefficient of friction surface 222.

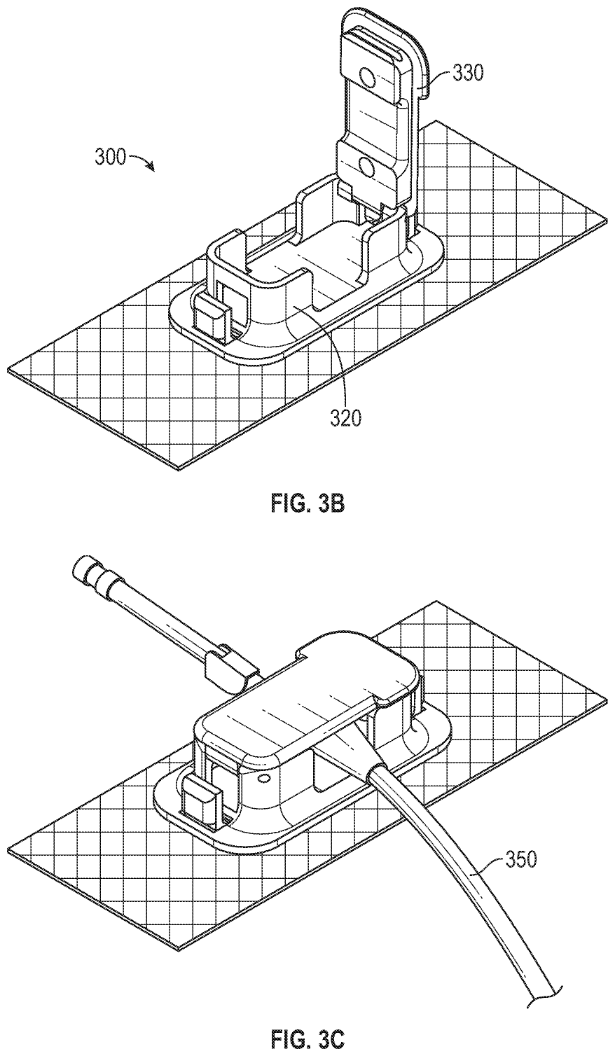

FIGS. 3A-3C shows a perspective view of a catheter stabilization device in use. FIG. 3A shows attachment article 340 that includes tabs 341 and 342, and base layer 310 of a hardgood article. In this embodiment, the base layer 310 is separate from the hardgood article and the hardgood article is detachable and re-attachable to the base layer 310. FIG. 3B shows hardgood article 300 attached to the base layer, the hardgood article includes holding member 320 and cover member 330. FIG. 3C shows the article of FIG. 3B with catheter and hub 350 attached.

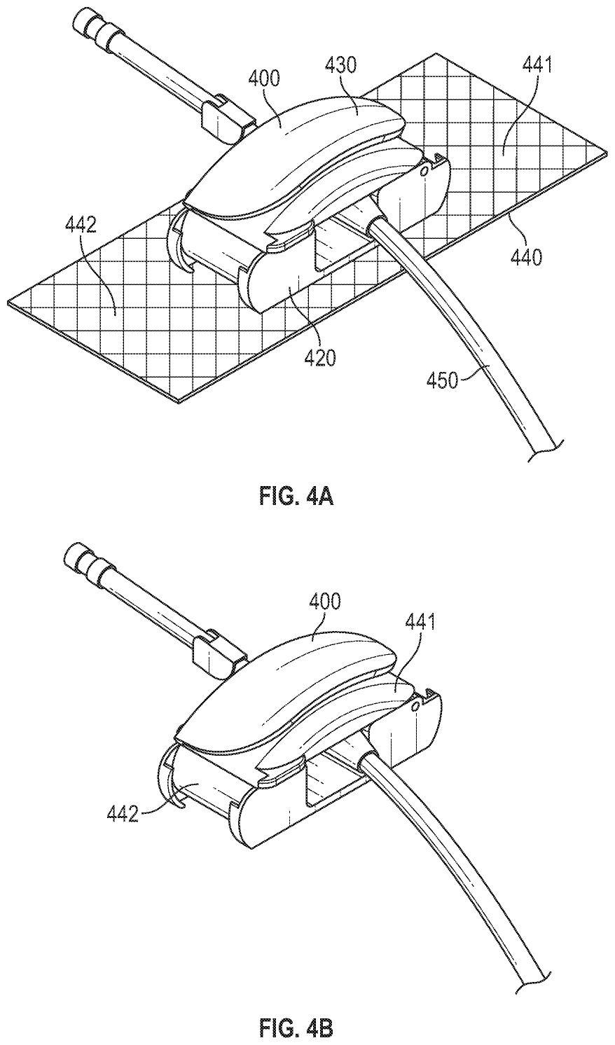

FIGS. 4A and 4B shows a perspective view of catheter stabilization device in use. FIG. 4A shows attachment article 440 that includes tabs 441 and 442 with attached hardgood article 400. Hardgood article 400 includes holding member 420 and cover member 430. Catheter and hub 450 is held by hardgood article 400. FIG. 4B shows the article of FIG. 4a with tabs 441 and 442 folded into hardgood article 400.

1. An attachment system for attaching a catheter to a patient, the attachment system comprising:
an adhesive attachment layer with a first major surface and a second major surface, wherein the first major surface comprises an adhesive surface suitable for attachment to skin of the patient; and
a polymeric hard good coupled with the adhesive attachment layer for holding a catheter, the polymeric hard good comprising:
a base layer with a first major surface and a second major surface, wherein the first major surface of the base layer is in contact with the second major surface of the adhesive attachment layer; and
a hinge, comprising:
a holding member, comprising:
a first major surface in contact with the second major surface of the base layer; and
a second major surface comprising a securement surface configured to hold a hub of the catheter, wherein the second major surface of the holding member comprises a conformable layer with a coefficient of friction of 0.5 or higher; and
a cover member coupled with the holding member, the cover member comprising a first major surface comprising a conformable layer and configured to contact at least a portion of the second major surface of the holding member when the hinge is closed, such that when closed the hinge holds the hub of the catheter.

2. The attachment system of claim 1, wherein the adhesive attachment layer comprises a multi-layer stretch removable member, comprising:
a multi-layer extensible backing substrate, comprising:
a first plastic skin layer with a first major surface and a second major surface;
a core elastomeric layer with a first major surface and a second major surface; and
a second plastic skin layer with a first major surface and a second major surface, wherein the core elastomeric layer is in contact with the second major surface of the first plastic skin layer, and the first major surface of the second plastic skin layer; and
a first adhesive layer in contact with the first plastic skin layer, wherein the first adhesive layer is configured for attachment to the skin of the patient; and
a second adhesive layer in contact with the second plastic skin layer, wherein the second adhesive layer attaches the multi-layer stretch removable member to the polymeric hard good.

3. The attachment system of claim 2, wherein the multi-layer extensible backing substrate comprises a material selected from the group consisting of polyester, polyurethane, (meth)acrylate, polyolefin, and combinations thereof.

4. The attachment system of claim 2, wherein the core elastomeric layer comprises at least one material selected from the group consisting of SEBS, SEPS, SIS, SBS, polyurethane, ethyl vinylacetate (EVA), ethyl methyl acrylate (EMA), ultra-low linear density polyethylene (ULLDPE), hydrogenated polypropylene, and combinations thereof.

5. The attachment system of claim 2, wherein the first plastic skin layer and the second plastic skin layer comprise a material selected from the group consisting of polypropylene, polyethylene, high density polyethylene (HDPE), low density polyethylene (LDPE), linear low-density polyethylene (LLDPE), a polyurethane, EVA, EMA, and combinations thereof.

6. The attachment system of claim 2, further comprising one or more tabs at a perimeter portion of the multi-layer extensible backing substrate.

7. The attachment system of claim 1, wherein the hinge is de-attachable from and re-attachable to the base layer.

8. The attachment system of claim 1, wherein the second major surface of the holding member comprises:
a first section;
a second edge section; and
a central section between the first edge section and the second edge section, wherein the central section comprises a depression relative to the first edge section and the second edge section.

9. The attachment system of claim 1, wherein at least a portion of the second major surface of the holding member comprises:
a first layer disposed on the holding member and comprising a conformable layer; and
a second layer disposed on the first layer and comprising a high coefficient of friction surface.

10. The attachment system of claim 9, wherein the first layer comprises a foam or a rubber polymer.

11. The attachment system of claim 1, wherein the securement surface of the second major surface of the holding member comprises a microstructured surface.

12. The attachment system of claim 1, wherein the first major surface of the cover member comprises a layer with a coefficient of friction of 0.5 or higher.

13.-15. (canceled)

16. The attachment system of claim 6, wherein the one or more tabs are folded into the polymeric hard good.

17. The attachment system of claim 1, further comprising a cover film covering at least a portion of the attachment system and a portion of the skin of the patient.

18. (canceled)

19. The medical construction of claim 1, wherein the securement surface of the second major surface of the holding member comprises a depression relative to one or more adjacent portions of the second major surface of the holding member.

20.-22. (canceled)
